(19) United States
(12) Patent Application Publication
(10) Pub. No.: US 2009/0043113 A1
(43) Pub. Date: Feb. 12, 2009

(54) Heteroacene compound, organic thin film including a heteroacene compound and electronic device including an organic thin film

(75) Inventors: Jeong Il Park, Seongnam-si (KR); Eun Jeong Jeong, Seongnam-si (KR); Sang Yoon Lee, Seoul (KR)

Correspondence Address: HARNESS, DICKEY & PIERCE, P.L.C., P.O. BOX 8910, RESTON, VA, 20195, US

(73) Assignee: Samsung Electronics Co., Ltd.

(21) Appl. No.: 12/081,671

(22) Filed: Apr. 18, 2008

(30) Foreign Application Priority Data
May 15, 2007 (KR) 10-2007-0047087

(51) Int. Cl. C07D 333/50

(52) U.S. Cl. 549/41

(57) ABSTRACT

A heteroacene compound, an organic thin film including a heteroacene compound and an electronic device including a thin film are provided. The heteroacene compound is a compound having six rings fused together in a compact planar structure. The compound may be used in an organic thin film and/or applied to electronic devices using a deposition process or a room-temperature solution process.

### PRIORITY STATEMENT

This non-provisional application claims the benefit of priority under U.S.C. § 119 from Korean Patent Application No. 10-2007-0047087, filed on May 15, 2007 with the Korean Intellectual Property Office (KIPO), the entire contents of which are herein incorporated by reference.

### BACKGROUND

1. Field

Example embodiments relate to a heteroacene compound, an organic thin film including the same and an electronic device including an organic thin film. Other example embodiments relate to a heteroacene compound, in which all six rings are fused together, an organic thin film including a heteroacene compound, and an electronic device that includes the thin film as a carrier transport layer.

2. Description of the Related Art

In general, flat display devices (e.g., liquid crystal displays, organic electroluminescent displays or the like) are provided with a variety of thin film transistors (TFTs) to drive them. A TFT may include a gate electrode, source and drain electrodes and/or a semiconductor layer that is activated in response to the operation of the gate electrode. Depending on the gate voltage applied, the semiconductor layer functions as a conductive channel for controlling the current between the source electrode and the drain electrode.

Materials that may be used for a channel layer of the TFT, in particular organic materials (e.g., pentacene, polythiophene and like compounds), are being thoroughly studied. Polymer or oligomer organic materials (e.g., F8T2 (dioctylfluorene-bithiophene), which is a polythiophene material) may used in a solution process (e.g., spin casting). However, problems such as low charge mobility and/or high off-state leakage current may occur. Although low-molecular-weight organic materials (e.g., pentacene) demonstrate a higher charge mobility of about 3.2 cm²/Vs−5.0 cm²/Vs or more, the use of low-molecular weight organic compounds requires costly equipment to form a thin film using vacuum deposition, which may be undesirable when preparing a film having a substantially large surface area.

The conventional art acknowledges the use of materials having a higher charge mobility such as dimeric bisbenzodithiophene (in which rings are fused in groups of three) and 2,7-diphenyl[1]-benzothieno[3,2-b]benzothiophene for channel layers.

### SUMMARY

Example embodiments relate to a heteroacene compound, an organic thin film including the same and an electronic device including an organic thin film. Other example embodiments relate to a heteroacene compound, in which all six rings are fused together, an organic thin film including a heteroacene compound, and an electronic device that includes the thin film as a carrier transport layer.

Example embodiments provide a low-molecular weight heteroacene compound, which has a compact planar structure in which all six rings are fused together, exhibiting higher charge mobility. Example embodiments provide a low-molecular weight heteroacene compound that may be used in a deposition process or a room-temperature solution process when applied to devices, increasing processability.

According to example embodiments, a low-molecular-weight heteroacene compound, which has a compact planar structure in which all six rings are fused together, is provided. The heteroacene compound may be represented by Formula (1) below:

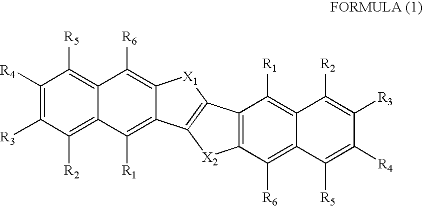

wherein X₁ and X₂ are each independently O, S, Se, Te, or N—R, in which R is selected from the group consisting of hydrogen, a C₁₋₁₂ alkyl group, a C₆₋₁₂ arylalkyl group, a C₆₋₁₂ aryl group, a C₁₋₁₂ alkoxy group, an acyl group, a sulfonyl group and a carbamate group. R₁, R₂, R₃, R₄, R₅, and R₆ are each independently selected from the group consisting of hydrogen, a substituted or unsubstituted C₁₋₃₀ alkyl group, a substituted or unsubstituted C₂₋₃₀ alkenyl group, a substituted or unsubstituted C₂₋₃₀ alkynyl group, a substituted or unsubstituted C₁₋₃₀ heteroalkyl group, a substituted or unsubstituted C₆₋₃₀ arylalkyl group, a substituted or unsubstituted C₂₋₃₀ heteroarylalkyl group, a substituted or unsubstituted C₆₋₃₀ cycloalkyl group, a substituted or unsubstituted C₂₋₃₀ heterocycloalkyl group, a substituted or unsubstituted C₆₋₃₀ aryl group and a substituted or unsubstituted C₂₋₃₀ heteroaryl group.

According to example embodiments, an organic thin film including a heteroacene compound is also provided.

According to yet other example embodiments, an electronic device including an organic thin film as a carrier transport layer is provided.

### BRIEF DESCRIPTION OF THE DRAWINGS

Example embodiments will be more clearly understood from the following detailed description taken in conjunction with the accompanying drawings. FIGS. 1-3 represent non-limiting, example embodiments as described herein.

FIG. 1 is an H-NMR graph of the heteroacene compound synthesized in Preparative Example 1 according to example embodiments;

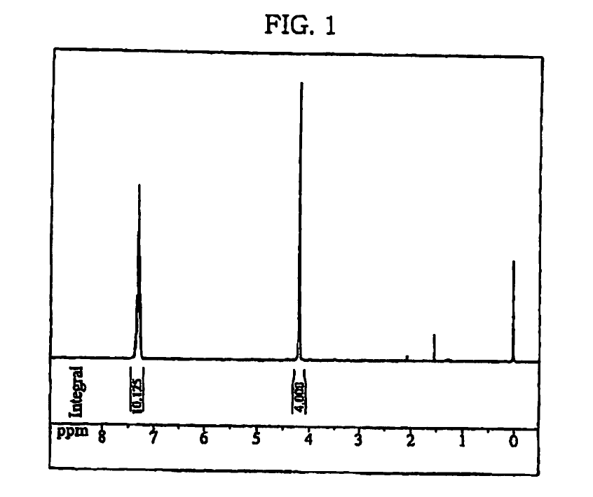

FIG. 2 is a C-NMR graph of the heteroacene compound synthesized in Preparative Example 1 according to example embodiments; and

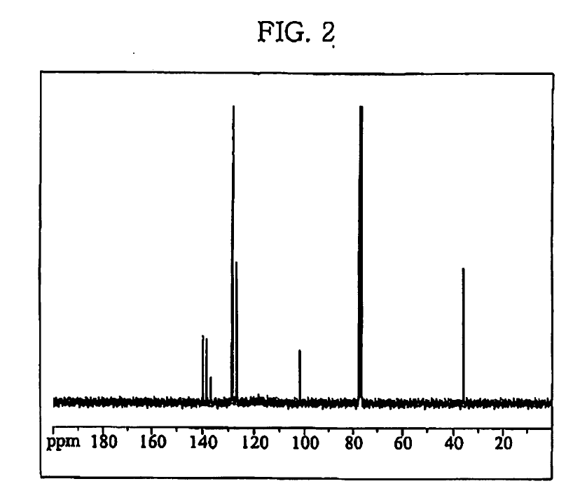

FIG. 3 is a graph illustrating the results of thermal analysis of the heteroacene compound synthesized in Preparative Example 1 according to example embodiments.

### DESCRIPTION OF EXAMPLE EMBODIMENTS

Various example embodiments will now be described more fully with reference to the accompanying drawings in which some example embodiments are shown.

Detailed illustrative embodiments are disclosed herein. However, specific structural and functional details disclosed herein are merely representative for purposes of describing example embodiments. This invention may, however, may be embodied in many alternate forms and should not be construed as limited to only example embodiments set forth herein.

Accordingly, while example embodiments are capable of various modifications and alternative forms, embodiments thereof are shown by way of example in the drawings and will herein be described in detail. It should be understood, however, that there is no intent to limit example embodiments to the particular forms disclosed, but on the contrary, example embodiments are to cover all modifications, equivalents, and alternatives falling within the scope of the invention.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term “and/or” includes any and all combinations of one or more of the associated listed items.

It will be understood that when an element is referred to as being “connected” or “coupled” to another element, it can be directly connected or coupled to the other element or intervening elements may be present In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the scope of example embodiments.

Spatially relative terms, such as “beneath,” “below,” “lower,” “above,” “upper” and the like, may be used herein for ease of description to describe one element or a relationship between a feature and another element or feature as illustrated in the figures. It will be understood that the spatially relative terms are intended to encompass dfferent orientations of the device in use or operation in addition to the orientation depicted in the Figures. For example, if the device in the figures is turned over, elements described as “below” or “beneath” other elements or features would then be oriented “above” the other elements or features. Thus, for example, the term “below” can encompass both an orientation which is above as well as below. The device may be otherwise oriented (rotated 90 degrees or viewed or referenced at other orientations) and the spatially relative descriptors used herein should be interpreted accordingly.

Unless otherwise defined, all terms (including technical and scientific terms) used herein have the same meaning as commonly understood by one of ordinary skill in the art to which example embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In order to more specifically describe example embodiments, various aspects will be described in detail with reference to the attached drawings. However, the present invention is not limited to example embodiments described.

Example embodiments relate to a heteroacene compound, an organic thin film including the same and an electronic device including an organic thin film. Other example embodiments relate to a heteroacene compound, in which all six rings are fused together, an organic thin film including a heteroacene compound, and an electronic device that includes the thin film as a carrier transport layer.

Example embodiments provide a heteroacene compound represented by Formula (1):

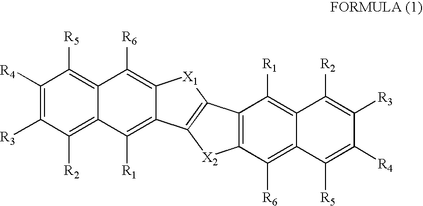

wherein X₁ and X₂ are each independently O, S, Se, Te, or N—R, in which R is selected from the group consisting of hydrogen, a C₁₋₁₂ alkyl group, a C₆₋₁₂ arylalkyl group, a C₆₋₁₂ aryl group, a C₁₋₁₂ alkoxy group, an acyl group, a sulfonyl group and a carbamate group. R₁, R₂, R₃, R₄, R₅, and R₆ are each independently selected from the group consisting of hydrogen, a substituted or unsubstituted C₁₋₃₀ alkyl group, a substituted or unsubstituted C₂₋₃₀ alkenyl group, a substituted or unsubstituted C₂₋₃₀ alkynyl group, a substituted or unsubstituted C₁₋₃₀ heteroalkyl group, a substituted or unsubstituted C arylalkyl group, a substituted or unsubstituted C₂₋₃₀ heteroarylalkyl group, a substituted or unsubstituted C₂₋₃₀ cycloalkyl group, a substituted or unsubstituted C₂₋₃ heterocycloalkyl group, a substituted or unsubstituted C₆₋₃₀ aryl group, and a substituted or unsubstituted C₂₋₃₀ heteroaryl group.

As represented by Formula (1), the heteroacene compound has a structure in which all six aromatic and heteroaromatic rings are fused together. If the compound having such a compact planar molecular structure is applied to devices, the oxidation potential may be more uniform and/or stable. Intermolecular packing and stacking of the heteroacene compound according to example embodiments may be more efficient, resulting in higher charge mobility. Because the heteroacene compound is easily synthesized, it may be used as a semiconductor material or an electron transport material.

The heteroacene compound may have a substituent (e.g., an alkyl group, an alkenyl group, or an aryl group) which may be introduced into the naphthalene rings at terminal portions thereof, increasing solubility of the heteroacene compound. As such, the compound may be used in a deposition process or in a room-temperature solution process. The heteroacene compound according to example embodiments may also be formed into a thin film having a substantially large surface area, providing more effective processability and workability.

In Formula 1, the alkyl group may be a linear or branched type chain (e.g., methyl, ethyl, propyl, iso-butyl, sec-butyl, tert-butyl, pentyl, iso-amyl, hexyl or the like). At least one hydrogen atom included in the alkyl group may be substituted with a predetermined substituent. An amount of the predetermined substituent varies depending on use and/or need. Examples of the substituent include, but are not limited to, a C₁₋₁₀ alkyl group, a C₂₋₁₀ alkenyl group, a C₂₋₁₀ alkynyl group, a C₆₋₁₂ aryl group, a C₂₋₁₂ heteroaryl group, a C₆₋₁₂ arylalkyl group, a halogen atom, a cyano group, an amino group, an amidino group, a nitro group, an amide group, a carbonyl group, a hydroxyl group, a sulfonyl group, a carbamate group and a C₁₋₁₀ alkoxy group.

The alkenyl group or alkynyl group has at least one carbon-carbon double bond or triple bond in the middle portion or the terminal portion of the alkyl group discussed above. Examples thereof include ethylene, propylene, butylene, hexylene and acetylene. At least one hydrogen atom of the alkenyl group or alkynyl group may be substituted with the same substituents as the alkyl group.

The heteroalkyl group may be a radical formed by replacing at least one carbon atom, or 1-5 carbon atoms, of the main chain of the alkyl group with a hetero atom (e.g., oxygen, sulfur, nitrogen, phosphorus or the like). At least one hydrogen atom of the heteroalkyl group may be substituted with the same substituents as the alkyl group.

The aryl group may be an aromatic carbocyclic system having at least one aromatic ring, the ring being attached or fused together through a pendent process. Examples of the aryl group include phenyl, naphthyl, tetrahydronaphthyl or the like. At least one hydrogen atom of the aryl group may be substituted with the same substituents as the alkyl group.

The arylalkyl group may be a radical formed by replacing at least one of the hydrogen atoms of the aryl group (discussed above) with a lower alkyl radical (e.g., methyl, ethyl, propyl or the like). The arylalkyl group may be benzyl, phenylethyl or the like. At least one hydrogen atom of the arylalkyl group may be substituted with the same substituents the alkyl group.

The cycloalkyl group may be a monovalent monocyclic system having 5-30 carbon atoms. The heterocycloalkyl group may be a 5- to 30-member monovalent monocyclic system having 1-3 hetero atoms selected from the group consisting of nitrogen (N), oxygen (O), phosphorus (P) and sulfur (S) with the remaining ring atoms being carbon (C). At least one of the hydrogen atoms of the cycloalkyl group or heterocycloalkyl group may be substituted with the same substituents as the alkyl group.

The heteroaryl group may be a 5- to 30-member aromatic ring system having 1-3 hetero atoms selected from the group consisting of nitrogen (N), oxygen (O), phosphorus (P) and sulfur (S) with the remaining ring atoms being carbon (C). The rings of the heteroaryl group may be attached or fused together through a pendent process. At least one of the hydrogen atoms of the heteroaryl group may be substituted with the same substituents as the alkyl group.

The heteroarylalkyl group may be a radical formed by replacing at least one of the hydrogen atoms of the heteroaryl group (discussed above) with a lower alkyl. At least one of the hydrogen atoms of the heteroarylalkyl group may be substituted with the same substituents as the alkyl group.

The alkoxy group may be a radical-O-alkyl, in which the alkyl is one of the groups discussed above. Examples thereof include methoxy, ethoxy, propoxy, iso-butyloxy, sec-butyloxy, pentyloxy, iso-amyloxy and hexyloxy. At least one of the hydrogen atoms of the alkoxy group may be substituted with the same substituents as the alkyl group.

The heteroacene compound may be a dinaphthothienothiophene derivative represented by Formula (2) below, in which R₁, R₂, R₃, R₄, R₅, and R₆ are each independently substituents that increase solvent solubility and/or exhibit higher charge mobility.

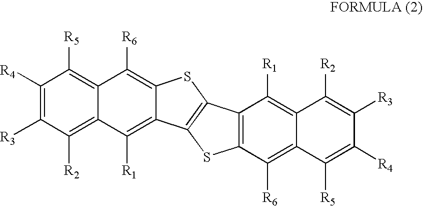

wherein R₁, R₂, R₃, R₄, R₅, and R₆ are each independently hydrogen, a C₁₋₁₀ alkyl group, a C₂₋₁₀ alkenyl group, a C₁₋₂ arylalkyl group or a C₁₋₁₂ aryl group.

Examples of the compound according to Formula (2) include, but are not limited to, the compounds represented by Formulas (3) to (6) below:

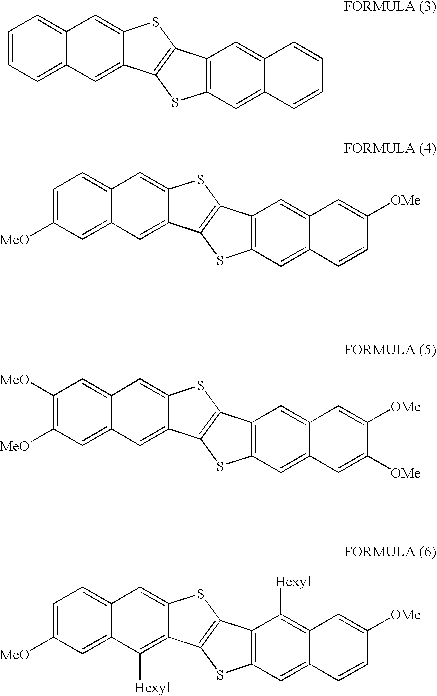

The heteroacene compound according to example embodiments may be synthesized using a conventional process for polymerizing aromatic or heteroaromatic compounds. The conventional process for polymerizing aromatic or heteroaromatic compounds may include a chemical or electrochemical oxidation synthesis process and a condensation process using an organic transition metal compound (e.g., including nickel or palladium). The heteroacene compound according to example embodiments may be synthesized according to Reaction (1) below, but example embodiments are not limited thereto.

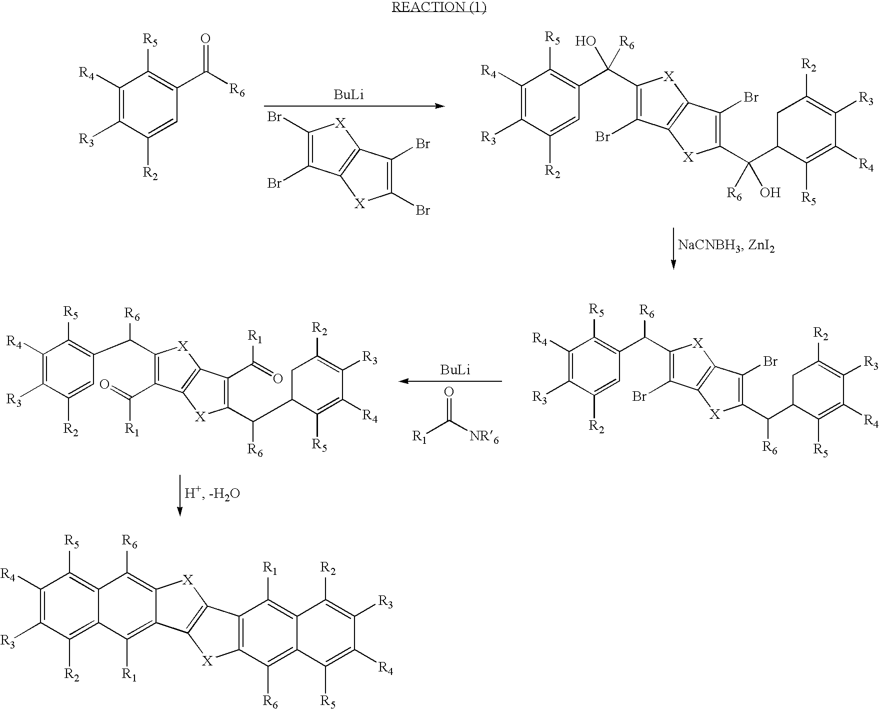

wherein X₁, X₂, R₁, R₂, R₃, R₄, R₅, and R₆ are each independently hydrogen, a C₁₋₁₀ alkyl group, a C₂₋₁₀ alkenyl group, a C₆₋₁₂ arylalkyl group or a C₆₋₁₂ aryl group.

The above reaction may be performed at a temperature ranging from −78° C. to room temperature in an ambient atmosphere or in a nitrogen atmosphere using a heteroaromatic ring compound substituted with a halogen atom (e.g., bromine) and/or lithium. In the reaction, the solvent may include toluene, dimethylformamide, N-methylpyrrolidinone and tetrahydrofuran. An acidic catalyst (e.g., Amberlyst 15) may be used as a dehydrating catalyst.

Tetrabromothienothiophene may be subjected to selective lithiation and to reaction with benzaldehyde, producing a dihydroxy compound. The dihydroxy compound may be dehydrated using borohydride as a reducing agent. The dehydrated dihydroxy compound may be subjected to lithiation and subsequently introduced to an aldehyde. The resulting compound may be dehydrated in the presence of an acidic catalyst such that both cyclization and aromatization occur, forming a low-molecular-weight compound with fused rings.

The molecular weight of the heteroacene compound may be in a range of 300 to 3000. The molecular weight of the heteroacene compound may be appropriately controlled depending on use and/or need.

The heteroacene compound of according to example embodiments may be used as a low-molecular-weight organic semiconductor material or as an electron transport material. Because the rings of the heteroacene compound are fused, the heteroacene compound exhibits higher charge mobility. Because the heteroacene compound includes thiophene, the heteroacene compound exhibits more stability. As such, the heteroacene compound may be subjected to a deposition process and/or a solution process. Due to the higher stability, the heteroacene compound may be used as a monomer for polymerization or doping.

Other example embodiments relate to an organic thin film including a heteroacene compound (as discussed above).

The organic thin film, including the heteroacene compound, may be used as a carrier transport layer (e.g., an organic semiconductor layer or a channel layer) of an electronic device. The electronic device including the thin film may exhibit higher processability and workability and/or increased electrical properties, including higher charge mobility.

The organic thin film may be formed by dissolving at least one heteroacene compound in an organic solvent and applying the solution on a substrate using a conventional deposition process or a conventional room-temperature solution process. After deposition or application of the solution, a heat treatment may be performed depending on the use, increasing the density and/or uniformity of the thin film.

The organic solvent may include at least one organic solvent selected from the group consisting of an aliphatic hydrocarbon solvent, an aromatic hydrocarbon solvent, a ketone-based solvent, an ether-based solvent, an acetate-based solvent, an alcohol-based solvent, an amide-based solvent, a silicon-based solvent and mixtures thereof.

The aliphatic hydrocarbon solvent may be at least one of hexane or heptane. The aromatic hydrocarbon solvent may be at least one of toluene, pyridine, quinoline, anisol, mesitylene and xylene. The ketone-based solvent may be at least one of methyl isobutyl ketone, 1-methyl-2-pyrrolidinone, cyclohexanone and acetone. The ether-based solvent may be at least one of tetrahydrofuran and isopropyl ether. The acetate-based solvent may be at least one of ethyl acetate, butyl acetate and propyleneglycol methyl ether acetate. The alcohol-based solvent may be at least one of isopropyl alcohol and butyl alcohol. The amide-based solvent may be at least one of dimethylacetamide and dimethylformamide.

The amount of the heteroacene compound dissolved in the organic solvent may be determined according to use. The amount of the heteroacene compound dissolved in the organic solvent may be within a range from about 0.01 wt % to about 50 wt % based on the total amount of the solution in order to increase solubility and/or applicability.

Examples of the organic thin film forming process include, but are not limited to, thermal evaporation, vacuum deposition, laser deposition, screen printing, printing, imprinting, spin casting, dipping, ink jetting, roll coating, flow coating, drop casting, spray coating, or roll printing. The heat treatment process may be conducted at a temperature ranging from 80° C. to 250° C. for a time period ranging from 1 min to 2 hours, but example embodiments are not limited thereto.

The thickness of the organic thin film may be in a range of 200 Å to 10,000 Å. However, example embodiments are not limited thereto. For example, the thickness of the organic thin film may be selected depending on use and/or need. The thickness of the organic thin film may be determined based on the type of compound and/or the type of solvent used.

Example embodiments relate to an electronic device including an organic thin film having a heteroacene compound.

Examples of the electronic device including the organic thin film as a carrier transport layer include, but are not limited to, TFTs, electroluminescent devices, photovoltaic devices, memory, sensors or the like. The organic thin film according to example embodiments may be applied to the electronic device using processes well-known in the art.

The TFT may include a substrate, a gate electrode, a gate insulating layer, source/drain electrodes and an organic semiconductor layer including an organic thin film according to example embodiments.

The TFT according to example embodiments may have a bottom gate structure, a semiconductor layer and a top gate structure. The bottom gate structure may include a substrate, a gate electrode formed on the substrate, a gate insulating layer formed on the gate electrode, source and drain electrodes formed on the gate insulating layer. The semiconductor layer may be formed on the gate insulating layer and the source/drain electrodes. The top gate structure may include a substrate, source/drain electrodes formed on the substrate, a semiconductor layer formed on the substrate and the source/drain electrodes, a gate insulating layer formed on the semiconductor layer and a gate electrode formed on the gate insulating layer. However, the example embodiments are not limited thereto, and the structure may be modified as long as the structure does not deviate from purpose of example embodiments.

The substrate is not particularly limited. Examples of substrates include, but are not limited to, silica, glass and plastic. Examples of the plastic substrate include, but are not limited to, polyethylene naphthalate, polyethylene terephthalate, polycarbonate, polyvinylbutyral, polyacrylate, polyimide, polynorbornene and polyethersulfone.

Materials well-known in the art may be used for the gate electrode and the source/drain electrodes. Example of materials suitable for the gate electrode and the source/drain electrodes include at least one selected from the group consisting of gold (Au), silver (Ag), aluminum (Al), nickel (Ni), molybdenum (Mo), tungsten (W), chromium (Cr), metal alloys thereof, metal oxides, conductive polymers and mixtures thereof.

The metal alloys may include a molybdenum/tungsten (Mo/W) alloy. The metal oxides may include indium tin oxide (ITO) and indium zinc oxide (IZO). The conductive polymers may include polythiophene, polyaniline, polyacetylene, polypyrrole, polyphenylene vinylene, and a mixture of PEDOT (polyethylenedioxythiophene) and PSS (polystyrenesulfonate).

The gate electrode may have a thickness of about 500 Å-2,000 Å. The source/drain electrodes may have a thickness of about 500 Å-2,000 Å. The electrodes may be formed in a patterning process. However, example embodiments are not limited thereto.

The gate insulating layer may be formed of any well-known material having a high dielectric constant. Examples of materials that may be used as the gate insulating layer include, but are not limited to, at least one selected from the group consisting of a ferroelectric insulator, an inorganic insulator and an organic insulator. The ferroelectric insulator may be selected from the group consisting of Ba_{0.33}Sr_{0.66}TiO₃ (BST), Al₂O₃, Ta₂O₅, La₂O₅, Y₂O₃, and TiO₂. The inorganic insulator may be selected from the group consisting of PbZr_{0.33}Ti_{0.66}O₃ (PZT), Bi₄Ti₃O₁₂, BaMgF₄, SrBi₂(TaNb)₂O₉, Ba(ZrTi)O₃(BZT), BaTiO₃, SrTiO₃, Bi₄Ti₃O₁₂, SiO₂, SiNₓ, and AlON. The organic insulator may be selected from the group consisting of polyimide, benzocyclobutene (BCB), parylene, polyacrylate, polyvinylalcohol and polyvinylphenol.

The gate insulating layer may have a thickness of about 1,000 Å-10,000 Å, but example embodiments are not limited thereto. The gate insulating layer may be formed in a patterning process.

A better understanding of example embodiments may be obtained in light of the following examples, which are set forth to illustrate, but are not to be construed as limiting.

### PREPARATIVE EXAMPLE 1

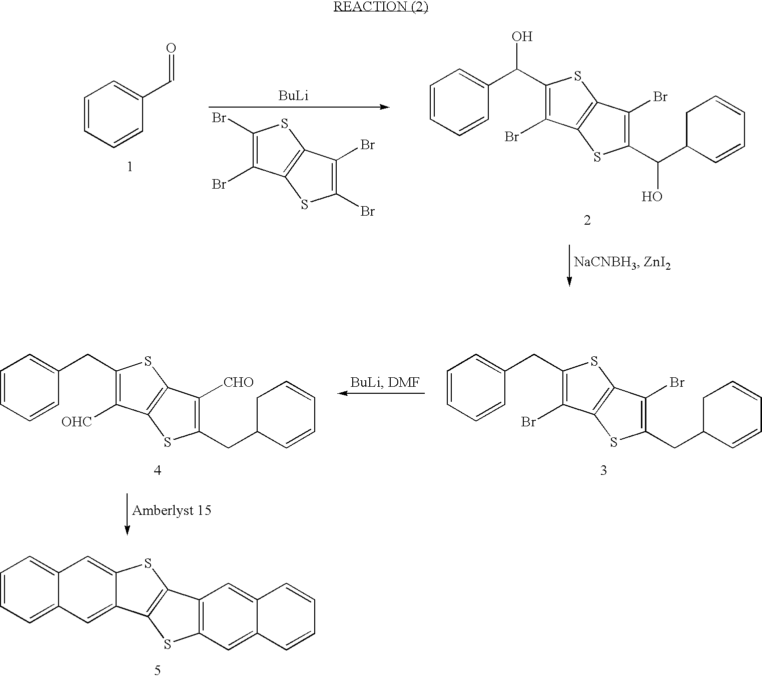

### Synthesis of Compound 2

Tetrabromothienothiophene was obtained by brominating thienothiophene using techniques developed by Iddon et al. (Lance S. Fuller, Brian Iddon, Kevin A. Smith J. Chem. Soc., Perkin Trans. 1, 1997, 34653470).

Tetrabromo-thieno[3,2-b]thiophene (2.0 g, 4.39 mmol) was dissolved in 50 ml of dry tetrahydrofuran (THF). The solution was cooled to −78° C. The cooled solution was slowly added with butyllithium (4 ml of 2.5 M in a hexane solution) and stirred for 30 min. Benzaldehyde (0.94 ml, 9.22 mmol) was slowly added in droplets. The solution was stirred overnight, allowing the temperature to gradually increase to room temperature. The solution was added with 50 ml of a saturated ammonium chloride solution and diluted with ethyl acetate. The organic layer was separated, dried and concentrated under reduced pressure, yielding compound 2 (a white solid). Compound 2 was used directly in the following reaction without additional purification or separation.

### Synthesis of Compound 3

Compound 2 (2.3 g, 4.6 mmol) was dissolved in 150 ml of dichloromethane and slowly added to ZnI2 (4.2 g, 13.2 mmol) and NaCNBH3 (3.6 g, 57.3 mmol). The mixture was stirred at room temperature for 24 hours and filtered using a celite pad. The filtrate was washed with a saturated ammonium chloride solution and water, dried over MgSO₄, and concentrated under reduced pressure, obtaining yellow oil. The oil was purified through silica chromatography, yielding compound 3. The following NMR results were obtained from compound 3: 1NMR (CDCl₃) δ 7.35-7.21 (m, 10H), 4.19 (s, 4H) and 13NMR (CDCl₃) δ 140, 138.5, 137, 128.8, 128.7, 127, 103, 36.

### Synthesis of Compound 4

A solution (10 ml) of butyllithium (1.73 mmol) in THF was cooled to −78° C. The solution (5 ml) of compound 3 (370 mg, 0.77 mmol) in THF was slowly added in droplets. The obtained solution was stirred at −78° C. for about 20 min. DMF (150 ml, 1.97 mmol) was added to the solution and stirred overnight. Water was added to the stirred solution to terminate the reaction. The reaction product was added to 30 ml of ether and washed with water and brine. The organic layer was dried over MgSO₄ and concentrated under reduced pressure, obtaining a colorless oil. The colorless oil was purified through silica chromatography, yielding compound 4. The following NMR results were obtained from compound 4: ¹NMR (CDCl₃) δ 10.3 (s, 2H), 7.36-7.20 (m, 10H), 4.21 (s, 4H).

### Synthesis of Compound 5

The compound 4 (200 mg) was dissolved in 10 ml of toluene and added to Amberlyst 15 (300 mg). The mixture was stirred and refluxed to remove water using a Dean-Stark trap. After about 24 hours, a beige solid was precipitated. The temperature was decreased to room temperature to precipitate Amberlyst 15, after which the supernatant was decanted and filtered, yielding compound 5 (a light yellow solid having a slight fluorescent blue sky color). The compound 5 was purified through sublimation in a high vacuum (<104 torr). Thermal analysis and gas chromatography-mass spectrometry (GC-MS) of the compound 5 was conducted (m.p. 420° C., GC-MS (M+) 340).

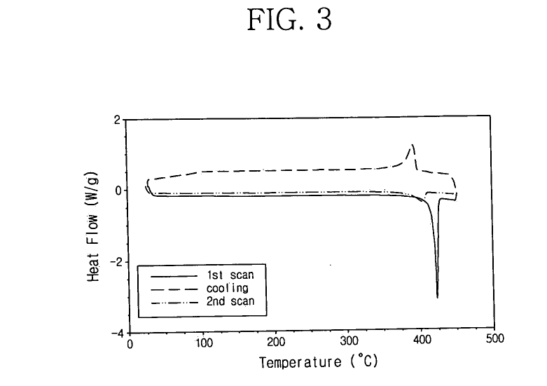

FIG. 3 is a graph illustrating the results of thermal analysis of the heteroacene compound synthesized in Preparative Example 1 according to example embodiments.

### EXAMPLE 1

On a washed glass substrate, aluminum (Al) for a gate electrode was deposited to a thickness of 1000 Å by sputtering. An organic and inorganic hybrid insulating material (a mixture of silane compound and titanium butoxide) was applied through spin casting and dried at 200° C. for 2 hours, forming a gate insulating layer having a thickness of 7000 Å. Gold (Au) for source/drain electrodes was deposited thereon to a thickness of 700 Å through thermal evaporation. The compound synthesized in Preparative Example 1 was deposited to a thickness of 700 Å by thermal evaporation to form an organic semiconductor layer, fabricating an organic thin film transistor (OTFT).

### COMPARATIVE EXAMPLE 1

An OTFT was fabricated in the same manner as in Example 1, except that bisbenzodithiophene (represented by Formula (7) below) was used instead of the compound according to Preparative Example 1.

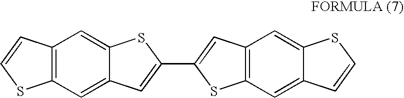

The charge mobility of the OTFTs fabricated in Example 1 and Comparative Example 1 was measured as follows. The results are given in Table 1 below.

The charge mobility was calculated from the current equations for the saturation region using the current transfer curve. The current equations for the saturation region were converted into a graph of (I_{SD})^{1/2} and V_{G}. The charge mobility was calculated based on the slope of the graph:

$${I}_{SD} = \frac{{WC}_{0}}{2 L} {μ ( {V}_{G} - {V}_{T} )}^{2}$$

$$\sqrt{{I}_{SD}} = \sqrt{\frac{μ {C}_{0} W}{2 L}} ( {V}_{G} - {V}_{T} )$$

$$slope = \sqrt{\frac{μ {C}_{0} W}{2 L}}$$

$${μ}_{FET} = {( slope )}^{2} \frac{2 L}{{C}_{0} W}$$

wherein I_{SD} is the source-drain current, μor μ_{FET} is the charge mobility, Cₒ is the oxide film capacitance, W is the channel width, L is the channel length, V_{G} is the gate voltage and V_{T} is the threshold voltage.

TABLE 1

| | CHARGE MOBILITY (cm²/Vs) |
|---|---|
| EXAMPLE 1 | 0.10 |
| COMPARATIVE EXAMPLE 1 | 0.02 |

As shown in Table 1, the OTFT using the heteroacene compound according to example embodiments has increased charge mobility.

As described above, example embodiments relate to a heteroacene compound, an organic thin film including the same, and an electronic device including a thin film. According to example embodiments, the heteroacene compound has a compact planar structure in which all six rings are fused together. As such, the heteroacene compound exhibits increased solvent solubility and/or processability. If the heteroacene compound according to example embodiments is used electronic devices, a deposition process or a room-temperature solution process may be performed. If the heteroacene compound according to example embodiments is used electronic devices, more efficient intermolecular packing and stacking occurs, resulting in increased electrical properties including higher charge mobility.

The foregoing is illustrative of example embodiments and is not to be construed as limiting thereof. Although a few example embodiments have been described, those skilled in the art will readily appreciate that many modifications are possible in example embodiments without materially departing from the novel teachings and advantages. Accordingly, all such modifications are intended to be included within the scope of this invention as defined in the claims. In the claims, means-plus-function clauses are intended to cover the structures described herein as performing the recited function, and not only structural equivalents but also equivalent structures. Therefore, it is to be understood that the foregoing is illustrative of various example embodiments and is not to be construed as limited to the specific embodiments disclosed, and that modifications to the disclosed embodiments, as well as other embodiments, are intended to be included within the scope of the appended claims.

What is claimed is:

1. A heteroacene compound, comprising a six-ring member structure represented by Formula (1): 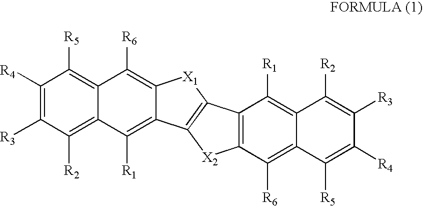
wherein X₁ and X₂ are each independently O, S, Se, Te, or N—R, in which R is selected from a group consisting of hydrogen, a C₁₋₁₂ alkyl group, a C₆₋₁₂ arylalkyl group, a C₆₋₁₂ aryl group, a C₁₋₁₂ alkoxy group, an acyl group, a sulfonyl group and a carbamate group, and
R₁, R₂, R₃, R₄, R₅, and R₆ are each independently selected from a group consisting of hydrogen, a substituted or unsubstituted C₁₋₃₀ alkyl group, a substituted or unsubstituted C₂₋₃₀ alkenyl group, a substituted or unsubstituted C₂₋₃₀ alkynyl group, a substituted or unsubstituted C₁₋₃₀ heteroalkyl group, a substituted or unsubstituted C₆₋₃₀ arylalkyl group, a substituted or unsubstituted C₂₋₃₀ heteroarylalkyl group, a substituted or unsubstituted C₅₋₂₀ cycloalkyl group, a substituted or unsubstituted C₂₋₃₀ heterocycloalkyl group, a substituted or unsubstituted C₆₋₃₀ aryl group, and a substituted or unsubstituted C₂₋₃₀ heteroaryl group.

2. The heteroacene compound as set forth in claim 1, wherein the alkyl group, the alkenyl group, the alkynyl group, the heteroalkyl group, the arylalkyl group, the heteroarylalkyl group, the cycloalkyl group, the heterocycloalkyl group, the aryl group and the heteroaryl group are each independently substituted with a substituent, which is at least one selected from a group consisting of a C₁₋₁₀ alkyl group, a C₂₋₁₀ alkenyl group, a C₂₋₁₀ alkynyl group, a C₆₋₁₂ aryl group, a C₂₋₁₂ heteroaryl group, a C₁₋₁₂ arylalkyl group, a halogen atom, a cyano group, an amino group, an amidino group, a nitro group, an amide group, a carbonyl group, a hydroxyl group, a sulfonyl group, a carbamate group and a C₁₋₁₀ alkoxy group.

3. The heteroacene compound as set forth in claim 1, wherein the heteroacene compound is a dinaphthothienothiophene derivative represented by Formula (2): 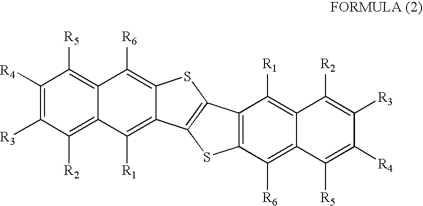
wherein R₁, R₂, R₃, R₄, R₅, and R₆ are each independently selected from a group consisting of hydrogen, a substituted or unsubstituted C₁₋₃₀ alkyl group, a substituted or unsubstituted C₂₋₃₀ alkenyl group, a substituted or unsubstituted C₂₋₃₀ alkynyl group, a substituted or unsubstituted C₁₋₃ heteroalkyl group, a substituted or unsubstituted C₁₋₃₀ arylalkyl group, a substituted or unsubstituted C₂₋₃₀ heteroarylalkyl group, a substituted or unsubstituted C₅₋₂₀ cycloalkyl group, a substituted or unsubstituted C₂₋₃₀ heterocycloalkyl group, a substituted or unsubstituted C₆₋₃₀ aryl group, and a substituted or unsubstituted C₂₋₃₀ heteroaryl group.

4. The heteroacene compound as set forth in claim 3, wherein the R₁, R₂, R₃, R₄, R₅, and R₆ are each independently selected from the group consisting of hydrogen, a C₁₋₁₀ alkyl group, a C₂₋₁₀ alkenyl group, a C₆₋₁₂ arylalkyl group and a C₆₋₁₂ aryl group.

5. The heteroacene compound as set forth in claim 3, wherein the dinaphthothienothiophene derivative of Formula (2) is represented by Formulas (3) to (6):

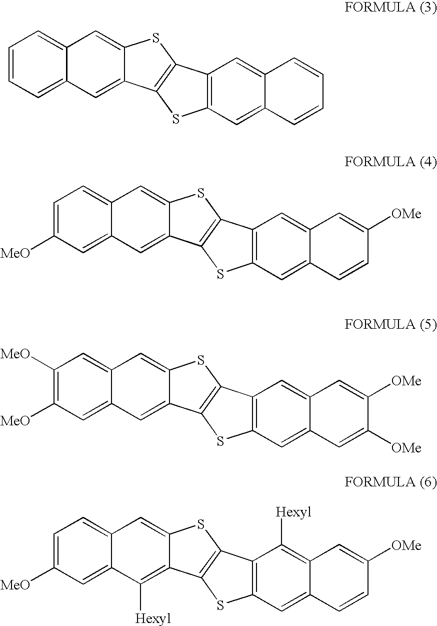

6. The heteroacene compound as set forth in claim 1, wherein the heteroacene compound has an average molecular weight ranging from 300 to 3000.

7. An organic thin film, comprising the heteroacene compound according to claim 1.

8. The organic thin film as set forth in claim 7, wherein the thin film is formed using at least one deposition process selected from the group consisting of thermal evaporabon, vacuum deposition, laser deposition, screen printing, printing, imprinting, spin casting, dipping, ink jetting, roll coating, flow coating, drop casting, spray coating and roll printing.

9. The organic thin film as set forth in claim 7, wherein the heteroacene compound is a dinaphthothienothiophene derivative represented by Formula (2): 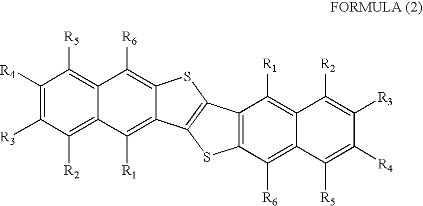
wherein R₁, R₂, R₃, R₄, R₅, and R₆ are each independently selected from a group consisting of hydrogen, a substituted or unsubstituted C₁₋₃₀ alkyl group, a substituted or unsubstituted C₂₋₃₀ alkenyl group, a substituted or unsubstituted C₂₋₃₀ alkynyl group, a substituted or unsubstituted C₁₋₃₀ heteroalkyl group, a substituted or unsubstituted C₁₋₃₀ arylalkyl group, a substituted or unsubstituted C₂₋₃₀ heteroarylalkyl group, a substituted or unsubstituted C₅₋₂₀ cycloalkyl group, a substituted or unsubstituted C₂₋₃₀ heterocycloalkyl group, a substituted or unsubstituted C₆₋₃₀ aryl group, and a substituted or unsubstituted C₂₋₃₀ heteroaryl group.

10. The organic thin film as set forth in claim 9, wherein the R₁, R₂, R₃, R₄, R₅, and R₆ are each independently selected from the group consisting of hydrogen, a C₁₋₁₀ alkyl group, a C₂₋₁₀ alkenyl group, a C₆₋₁₂ arylalkyl group and a C₆₋₁₂ aryl group.

11. The organic thin film as set forth in claim 9, wherein the dinaphthothienothiophene derivative of Formula (2) is represented by Formulas (3) to (6):

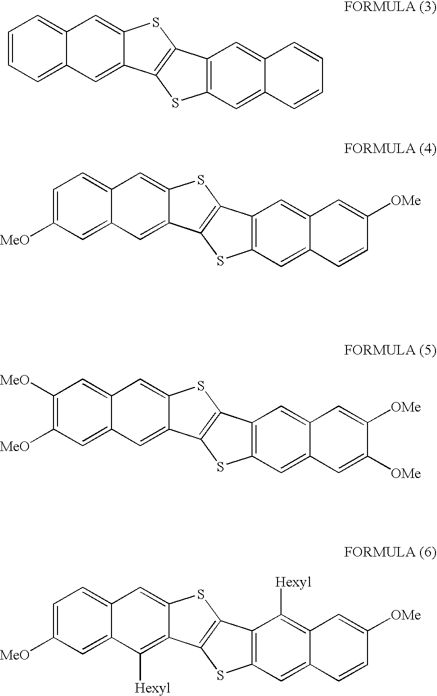

12. The organic thin film as set forth in claim 7, wherein the heteroacene compound has an average molecular weight ranging from 300 to 3000.

13. An electronic device, comprising the organic thin film according to claim 7.

14. The electronic device as set forth in claim 13, wherein the thin film is formed using at least one deposition process selected from the group consisting of thermal evaporation, vacuum deposition, laser deposition, screen printing, printing, imprinting, spin casting, dipping, ink jetting, roll coating, flow coating, drop casting, spray coating and roll printing.

15. The electronic device as set forth in claim 13, wherein the electronic device is a device selected from the group consisting of a thin film transistor, an electroluminescent device, a photovoltaic device, memory and a sensor.

16. The electronic device as set forth in claim 15, wherein the thin film is formed using at least one deposition process selected from the group consisting of thermal evaporation, vacuum deposition, laser deposition, screen printing, printing, imprinting, spin casting, dipping, ink jefting, roll coating, flow coating, drop casting, spray coating and roll printing.

17. The electronic device as set forth in claim 13, wherein the heteroacene compound is a dinaphthothienothiophene derivative represented by Formula (2): 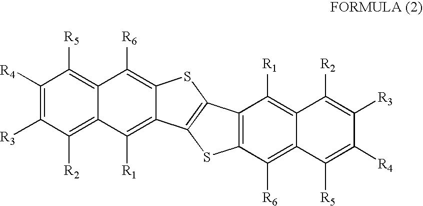
wherein R₁, R₂, R₃, R₄, R₅, and Rr are each independently selected from a group consisting of hydrogen, a substituted or unsubstituted C₁₋₃₀ alkyl group, a substituted or unsubstituted C₅₋₂₀ alkenyl group, a substituted or unsubstituted C₂₋₃₀ alkynyl group, a substituted or unsubstituted C₁₋₃₀ heteroalkyl group, a substituted or unsubstituted C₆₋₃₀ arylalkyl group, a substituted or unsubstituted C₂₋₃₀ heteroarylalkyl group, a substituted or unsubstituted C₅₋₂₀ cycloalkyl group, a substituted or unsubstituted C₂₋₃₀ heterocycloalkyl group, a substituted or unsubstituted C₆₋₃₀ aryl group, and a substituted or unsubstituted C₂₋₃₀ heteroaryl group.

18. The electronic device as set forth in claim 17, wherein the R₁, R₂, R₃, R₄, R₅, and R₆ are each independently selected from the group consisting of hydrogen, a C₁₋₁₀ alkyl group, a C₂₋₁₀ alkenyl group, a C₆₋₁₂ arylalkyl group and a C₆₋₁₂ aryl group.

19. The electronic device as set forth in claim 17, wherein the dinaphthothienothiophene derivative of Formula (2) is represented by Formulas (3) to (6):

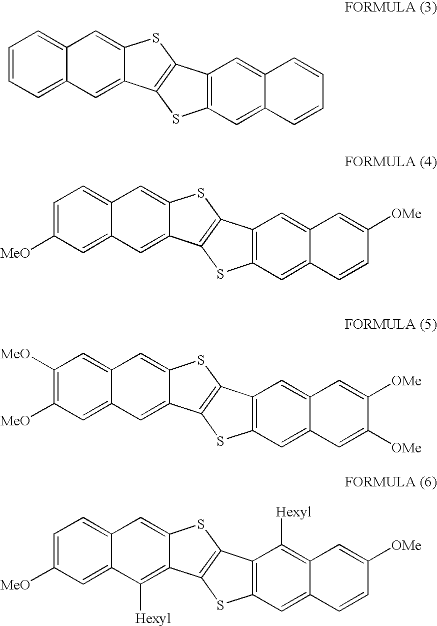

20. The electronic device as set forth in claim 13, wherein the heteroacene compound has an average molecular weight ranging from 300 to 3000.
